(19) United States
(12) Patent Application Publication
(10) Pub. No.: US 2006/0129165 A1
(43) Pub. Date: Jun. 15, 2006

(54) Gel based laparoscopic trocar

(75) Inventors: John K. Edoga, North Beach, NJ (US); Thierry Richard, Florham Park, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Edrich Health Technologies, Inc., Morristown, NJ (US)

(21) Appl. No.: 11/283,040

(22) Filed: Nov. 18, 2005

Related U.S. Application Data
(60) Provisional application No. 60/630,595, filed on Nov. 24, 2004.

(51) Int. Cl. A61F 11/00

(52) U.S. Cl. 606/108; 604/174

(57) ABSTRACT

A gel based laparoscopic trocar having a shaft with upper and lower portions, the shaft having an interior dimension, a rigid gel casing surrounding the upper portion of the shaft, at least two resilient gel filled membranes filling the interior dimension of the upper portion of the shaft, a plurality of petals surrounding the external portion of the lower portion of the shaft, the petals having a first condition in which the petals are elongated along the length of the shaft and a second condition in which portions of the petals are permitted to curl upward and away from the shaft, and a release mechanism associated with the petals, the release mechanism adapted to permit the petals to move from the first condition to the second condition upon actuation. The petals may include a memory metal having a natural condition relating to the second condition of the petals.

### CROSS-REFERENCE TO RELATED APPLICATIONS

This application claims the benefit of the filing date of U.S. Provisional Patent Application No. 60/630,595 filed Nov. 24, 2004, the disclosure of which is hereby incorporated herein by reference.

### BACKGROUND OF THE INVENTION

During many laparoscopic procedures, some of which are already in general clinical use and others of which are soon to be introduced, it is necessary to introduce medical devices for various purposes, such as rigid staplers used to perform anastomoses. Currently available laparoscopic trocars are not large enough to permit entry of such instruments. Further, the valve systems of presently available trocars are not adaptable for this purpose, as gasses used to swell the bodily cavity may readily escape. The shafts of present trocars are also not flexible, and therefore do not accommodate the aforementioned rigid instruments which frequently are curved or may need to be moved about and therefore require a somewhat permissive range of motion.

Surgeons therefore presently resort to introducing such instruments through surgical openings in the abdominal wall. In doing so, penetrating towel clips may be used to close the opening tightly around the shafts of the instruments being introduced into the body to reduce the rate of pneumoperitoneum loss.

Although practiced, this method is only partially successful. It has been found that insufflated CO₂ may escape, resulting in the reduction in the size of the operative space and attendant difficulties in achieving the exposure needed for proper execution of the surgical maneuver.

Furthermore in cases where a bowel anastomosis is performed with a stapler, for example during a gastrojejunostomy for gastric bypass operations or during any endoluminal intervention in a contaminated space such as a cyst gastrostomy for pancreatic cysts, the eventual removal of the instrument which has become contaminated in the process naturally results in the inoculation of vast numbers of bacteria and other pathogens into the body through the abdominal wall.

The present invention seeks to address these and other concerns.

### SUMMARY OF THE INVENTION

The present invention overcomes the shortcomings of the prior art by providing, in various combinations, a gel based laparoscopic trocar having features which enable the trocar to be used with a various array of instruments while preventing loss of pneumosperitoneum

In accordance with certain aspects of the present invention, a gel based laparoscopic trocar may comprise a bivalved gel plug contained within a semi-rigid plastic casing, the casing adapted to penetrate an opening formed in a bodily cavity. The internal bivalved gel plug may yield easily to permit penetration by a rigid instrument, yet may conform to the shape of the instrument to seal against the instrument and thereby prevent leakage of CO₂ from within the body cavity. The gel based laparoscopic trocar may also comprise a splitable sheath extending from the semi-rigid plastic casing. The sheath may be embedded with strips of memory alloy designed to curl once released by a pull wire system such that the curled portion will curl against the underside of the bodily wall surrounding the body cavity to squeeze a portion of the rigid gel casing against the exterior of the bodily wall.

In accordance with one aspect of the present invention, a gel based laparoscopic trocar may comprise a shaft having an upper portion and a lower portion, the shaft having an interior dimension, a rigid gel casing surrounding the upper portion of the shaft, at least two resilient gel filled membranes filling the interior dimension of the upper portion of the shaft, a plurality of petals surrounding the external portion of the lower portion of the shaft, the petals having a first condition in which the petals are elongated along the length of the shaft and a second condition in which portions of the petals are permitted to curl upward and away from the shaft, and a release mechanism associated with the petals, the release mechanism adapted to permit the petals to move from the first condition to the second condition upon actuation.

The petals may include a memory metal having a natural condition relating to the second condition of the petals.

The shaft may be flexible.

The shaft may be cylindrical and the dimension may be a diameter. The diameter may be between 1 cm and 12 cm.

The second portion of the shaft may be adapted to be placed through an opening formed in an abdominal wall of a patient such that the petals may pinch the abdominal wall between the rigid casing and the petals to hold the gel based laparoscopic trocar in place.

A surgical instrument may be inserted between the gel filled membranes and through the shaft, whereby the gel filled membranes may seal against the surgical instrument.

The trocar may further comprise a plastic membrane within the shaft, the plastic membrane being penetrable by a surgical instrument. The plastic membrane may seal against a surgical instrument penetrating therethrough.

The release mechanism may be a pull tie system.

In accordance with a further embodiment, a surgical access port may comprise a shaft having a first portion and a second portion, the first portion filled with at least two gel filled membranes, a casing surrounding the first portion of the shaft, a plurality of petals surrounding the second portion of the shaft, the petals having a first position in which the petals are elongated along the length of the second portion of the shaft and a second position in which the petals are permitted to curl away from the second portion of the shaft toward the casing, wherein the second portion of the shaft is adapted to be placed through an opening formed in an abdominal wall of a patient such that the petals may compress the abdominal wall against the casing to hold the surgical access port in place.

The petals may include a memory metal having a natural condition relating to the second position of the petals.

The shaft may be flexible.

The shaft may be cylindrical and the dimension may be a diameter, the diameter being between 1 cm and 12 cm.

A surgical instrument may be inserted between the at least two gel filled membranes and through the shaft, whereby the at least two gel filled membranes seal against the surgical instrument.

The port may further comprise a release mechanism, the release mechanism adapted to permit the petals to move from the first position to the second position.

The surgical access port may further comprise a plastic membrane within the shaft, the plastic membrane being penetrable by a surgical instrument. The plastic membrane may seal against a surgical instrument penetrating therethrough.

In accordance with another aspect of the present invention, a method of inserting a gel based laparoscopic trocar having a shaft with an upper portion and a lower portion, a rigid gel casing surrounding the upper portion of the shaft, at least two resilient gel filled membranes filling the first portion of the shaft, a plurality of petals surrounding the outside of the lower portion of the shaft, the petals having a first condition in which the petals are elongated along the length of the shaft and a second condition in which portions of the petals are permitted to curl upward and away from the shaft, the method may comprise the steps of forming a surgical incision in a patient, spreading the surgical incision, and inserting the lower portion of the gel based laparoscopic trocar into the incision.

The step of inserting may force the gel based laparoscopic trocar into the incision such that the gel casing abuts an exterior portion of the incision.

The method may further comprise the step of moving the petals from the first condition to the second condition such that the petals abut an interior portion of the incision.

### BRIEF DESCRIPTION OF THE DRAWINGS

The subject matter regarded as the invention is particularly pointed out and distinctly claimed in the concluding portion of the specification. The invention, however, both as to organization and methods of operation, together with features objects, and advantages thereof, may be best understood by reference to the following detailed description when read with the accompanying drawings in which:

FIG. 1 depicts a top plan view of a gel based laparoscopic trocar in accordance with a first embodiment of the present invention;

FIG. 2 depicts a side view of the gel based laparoscopic trocar of FIG. 1, prior to release of the pull ties;

FIG. 3 depicts a cross-sectional side view of the gel based laparoscopic trocar of FIG. 1, prior to release of the pull ties; and,

FIG. 4 depicts a side view of the gel based laparoscopic trocar of FIG. 1 installed through an abdominal wall after release of the pull ties.

### DESCRIPTION OF THE INVENTION

As previously discussed, existing laparoscopic trocars are unsuitable for use with many surgical instruments, particularly surgical staplers. In accordance with certain aspects of the present invention, an inventive gel based laparoscopic trocar solves this problem. The gel based laparoscopic trocar may comprise a bivalved gel plug contained within a semi-rigid plastic casing, the casing being adapted to penetrate an opening in a bodily cavity, particularly the abdomen. The internal bivalved gel plug may be resilient and yield upon entry to permit penetration by a rigid instrument, yet may conform to the shape of the instrument to seal against the instrument and thereby prevent leakage of CO₂ from within the body cavity once the instrument is inserted. The gel based laparoscopic trocar may also comprise a splitable sheath extending from the semi-rigid plastic casing. The sheath may be embedded with strips of memory alloy designed to curl once released by a retaining system such that the curled portion will abut the underside of the bodily wall surrounding the body cavity to squeeze a portion of the rigid gel casing against the exterior of the bodily wall, thus holding the trocar in position.

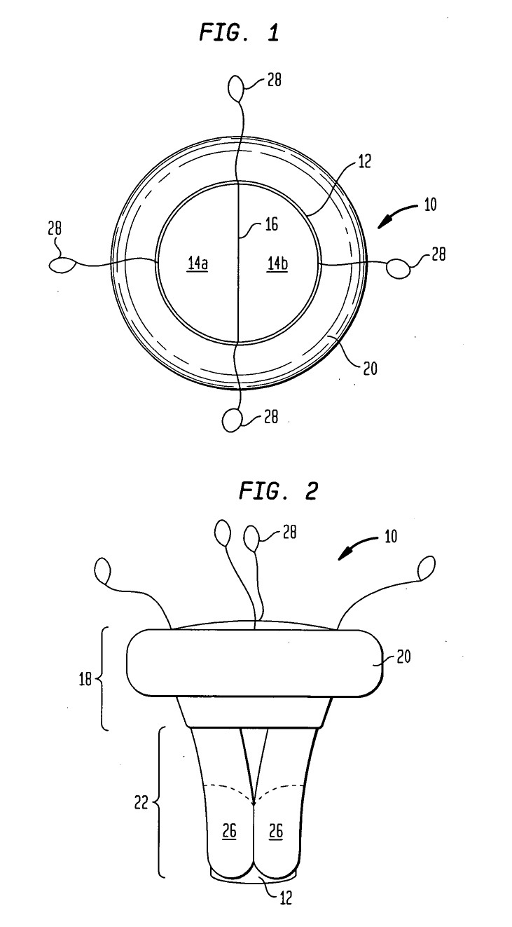

As shown in FIG. 1, a top view of a gel based laparoscopic trocar in accordance with certain aspects of the present invention, a gel based laparoscopic trocar 10 may comprise a flexible shaft 12 with two half cylinder segments, 14a, 14b filling the interior diameter thereof. The half-cylinder segments 14a, 14b typically comprise a resilient gel filled flexible membrane, such that the two segments are air tight against each other at their intersection 16 in the default position. The half-cylinder segments 14a, 14b are also preferably resilient such that they may be separated at their intersection 16 by force, yet will return to their default position to seal against a penetrating object. Accordingly, when a medical instrument is inserted into the intersection 16 of the half-cylinder segments 14a, 14b, the half-cylinder segments will yield to permit penetration, but will seal against the instrument once inserted.

It will be appreciated that greater than two gel filled membranes may also be provided. For example, three, four, or even a greater number of segments may be utilized. In such cases, there may be a plurality of intersections through which surgical objects may be inserted.

The shaft 12 is typically manufactured from plastics that are relatively firm, yet flexible. The half-cylinder segments 14a, 14b, may be made from various gel materials, and may include an external skin or membrane. Preferably each of the components of the trocar 10, whether previously mentioned or to be discussed, are biocompatible.

As shown in FIG. 2, a side view of the gel based laparoscopic trocar 10 of FIG. 1, the upper portion 18 of the shaft 12 may be externally surrounded by a rigid gel casing 20. A portion of the rigid gel casing 20 is designed to be of a much greater diameter than the shaft 12, such that the gel casing may be utilized to form a collar which prevents the trocar 10 from being pushed completely through a bodily opening sized to accept the shaft, as will be discussed.

The rigid gel casing 20 may comprise and outer shell formed from various metals, and may be filled with a viscous gel. Alternatively, the casing may be formed completely from plastic or metal, or may be formed entirely from a thick gel. Typically, the firmness of the rigid gel casing 20 is much greater than the firmness of the gel filled half-cylinder segments 14a, 14b.

A lower portion 22 of the shaft 12 may be surrounded by a splitable sheath 24, having a plurality of petals 26. Each of the petals 26 may be tied into a first position, shown in FIG. 2, with pull ties 28 such that the petals are adjacent the shaft 12. In this regard, the petals 26 are relatively straight and may be held firmly against the shaft 12. In this position, the gel based laparoscopic trocar 10 may be readily pushed through a bodily opening, such that the rigid gel casing 20 abuts the bodily opening.

The petals 28 may each contain at least one embedded strip of memory alloy such as Nitinol (not shown) which is curled in its natural state (such that the Nitinol is not in its natural state when the petals 28 are in the position shown in FIG. 2). This curled Nitinol forces the petals 26 to curl outwardly when the pull ties 28 are pulled, thus releasing the petals and allowing the Nitinol to return to its natural state. In other embodiments, the petals 26 may be glued in place. The pull ties 14 may then be utilized to sever the glue, such that the petals 26 are free to curl. Other release mechanisms in addition to pull ties 28 may also be utilized, so long as they serve to restrain the petals 26 in one mode of operation, and release the petals in another.

FIG. 3 depicts a cross-sectional side view of the gel based laparoscopic trocar of FIG. 1, prior to release of the pull ties 28. As shown, the gel based laparoscopic trocar 10 may further comprise a perforated plastic membrane 30 in the upper portion 18 thereof, just below the level of the half-cylinder segments 14a, 14b. The perforated plastic membrane 30 may include a perforation 32, which is adapted to rupture upon entry of a surgical instrument. The membrane 30 serves as an additional barrier guarding against the escape of insufflated CO₂. In this regard, it is preferred that the membrane 30 be formed from an elastic material, such that the membrane will seal against an inserted instrument.

As previously discussed, the gel based laparoscopic trocar 10 may be inserted into a surgical opening formed in a body, such as through an abdominal wall. Preferably, the surgical opening is sized approximately of equal dimension to that of the shaft 12 diameter, such that the gel based laparoscopic trocar 10 will be tight against the surgical opening throughout it's 360 degree exterior.

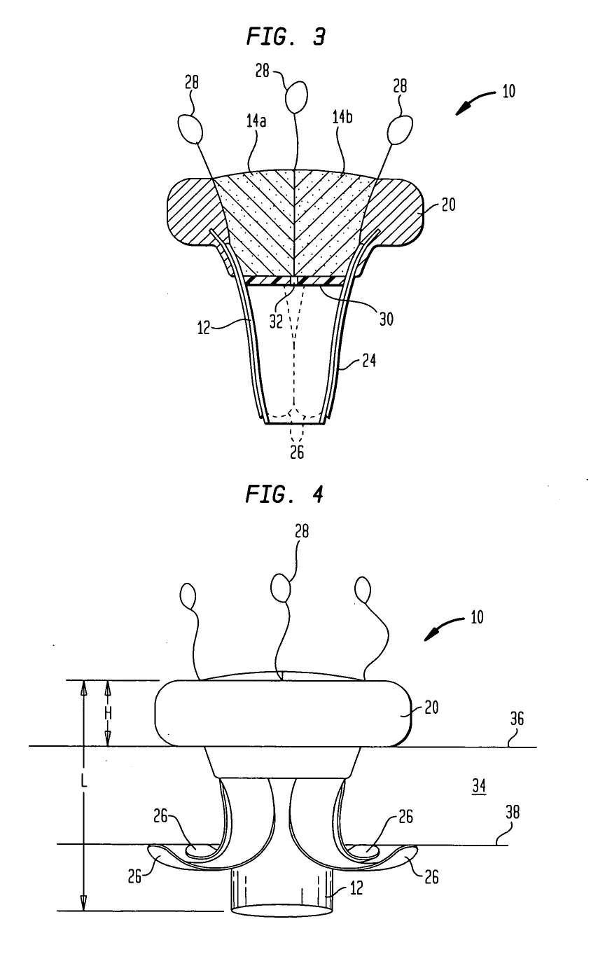

FIG. 4 depicts a side view of the gel based laparoscopic trocar of FIG. 1 installed through an abdominal wall 34. As previously stated, the gel based trocar 10 may be inserted into a surgical opening formed in the abdominal wall 34 while in the position shown in FIG. 2, with the petals 26 held adjacent to the shaft 12 by release mechanisms, such as pull ties 28. Preferably, the gel based laparoscopic trocar 10 is inserted until the rigid gel casing 20 abuts the exterior portion 36 of the abdominal wall 34. Once so inserted, the surgeon may actuate the release mechanism to permit the petals 26 to curl.

This curling continues until the inner surface 38 of the abdominal wall 36 is encountered by the petals 26, as shown in FIG. 4. The trocar 10 is therefore retained as the rigid gel casing 20 and petals 26 squeeze each other against the abdominal wall 34, and it is unlikely to be dislodged by usual surgical activity.

The curling action of the petals 26 allows the gel based laparoscopic trocar 10 to be utilized for patients of various girth. For example, in a thin patient, the petals 26 will curl a larger distance than in a more obese patient, where the abdominal wall 34 is thicker. In this regard, the gel based laparoscopic trocar 10 may be sized such that a single unit will be capable of use for the vast majority of patients, even though multiple sizes may also be manufactured, depending on the need.

When the petals 26 are permitted to peel apart and curl in the fashion shown in FIG. 4, the internal cylindrical shaft 12 is exposed. Instruments or other objects may be introduced into, and removed from, the abdomen through the shaft 12 without fear of contact with or contamination of the abdominal wall 36 as the instruments are completely contained within the shaft. The shaft 12 is preferably compliant, such that instruments or other objects placed therein may be shifted, bent or otherwise manipulated freely.

The shaft 12 within the gel based laparoscopic trocar 10 may be of any diameter ranging from 1 cm for the introduction of standard laparoscopic instruments such as a laparoscope telescope, 4 cm for the introduction of laparoscopic EEA staplers and vascular staplers, or up to 12 cm or more for the introduction of a large male hand. It is preferred that the overall length L of the gel based laparoscopic trocar 10 be approximately 10 cm, such that the trocar is long enough to extend into the thickness of the patient's abdomen. Of course, this dimension may be adjusted for different sized trocars, or for different sized intended patients, for example obese patients. Of the overall length, a portion will be taken up by the height H of the rigid gel casing 20. This height H may vary, as it is exposed above the patient's skin.

In order to insert the gel based laparoscopic trocar 10 into the surgical opening, a surgeon may form the surgical opening by making a surgical cut. The surgeon may then spread open the surgical cut with his or her hands to open the incision. The gel based laparoscopic trocar 10 may then be inserted such that the casing 20 abuts the exterior portion of the opening. The surgeon may then operate the release mechanism to allow the petals 26 to curl against the inside portion 38 of the opening, thus preventing the trocar 10 from being pulled out.

In accordance with other aspects of the present invention, a gel based laparoscopic trocar may also incorporate a retractable blade mechanism, such that the trocar itself is capable of making a surgical cut into the bodily cavity. Devices incorporating retractable blades are known in the industry, and the teachings of which may be combined with the present teachings to form such a trocar. Generally, a small incision may be made into the patient in the conventional manner. The blade of the gel based laparoscopic trocar may then be used to lengthen the incision, whereby the actual trocar may follow.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A gel based laparoscopic trocar comprising:
a shaft having an upper portion and a lower portion, said shaft having an interior dimension;
a rigid gel casing surrounding said upper portion of said shaft;
at least two resilient gel filled membranes filling said interior dimension of said upper portion of said shaft;
a plurality of petals surrounding the external portion of said lower portion of said shaft, said petals having a first condition in which said petals are elongated along the length of said shaft and a second condition in which portions of said petals are permitted to curl upward and away from said shaft; and,
a release mechanism associated with said petals, said release mechanism adapted to permit said petals to move from said first condition to said second condition upon actuation.

2. The gel based laparoscopic trocar of claim 1, wherein said petals include a memory metal having a natural condition relating to said second condition of said petals.

3. The gel based laparoscopic trocar of claim 1, wherein said shaft is flexible.

4. The gel based laparoscopic trocar of claim 1, wherein said shaft is cylindrical and said dimension is a diameter.

5. The gel based laparoscopic trocar of claim 4, wherein said diameter is between 1 cm and 12 cm.

6. The gel based laparoscopic trocar of claim 1, wherein said second portion of said shaft is adapted to be placed through an opening formed in an abdominal wall of a patient such that said petals may pinch the abdominal wall between the rigid casing and the petals to hold the gel based laparoscopic trocar in place.

7. The gel based laparoscopic trocar of claim 1, wherein a surgical instrument may be inserted between said gel filled membranes and through said shaft, whereby said gel filled membranes seal against the surgical instrument.

8. The gel based laparoscopic trocar of claim 1, wherein said trocar further comprises a plastic membrane within said shaft, said plastic membrane being penetrable by a surgical instrument.

9. The gel based laparoscopic trocar of claim 8, wherein said plastic membrane seals against a surgical instrument penetrating therethrough.

10. The gel based laparoscopic trocar of claim 1, wherein said release mechanism is a pull tie system.

11. A surgical access port comprising:
a shaft having a first portion and a second portion, said first portion filled with at least two gel filled membranes;
a casing surrounding said first portion of said shaft;
a plurality of petals surrounding said second portion of said shaft, said petals having a first position in which said petals are elongated along the length of said second portion of said shaft and a second position in which said petals are permitted to curl away from said second portion of said shaft toward said casing;
wherein said second portion of said shaft is adapted to be placed through an opening formed in an abdominal wall of a patient such that said petals may compress the abdominal wall against the casing to hold the surgical access port in place.

12. The surgical access port of claim 11, wherein said petals include a memory metal having a natural condition relating to said second position of said petals.

13. The surgical access port of claim 11, wherein said shaft is flexible.

14. The surgical access port of claim 11, wherein said shaft is cylindrical and said dimension is a diameter, said diameter being between 1 cm and 12 cm.

15. The surgical access port of claim 11, wherein a surgical instrument may be inserted between said at least two gel filled membranes and through said shaft, whereby said at least two gel filled membranes seal against the surgical instrument.

16. The surgical access port of claim 11, wherein said port further comprises a release mechanism, said release mechanism adapted to permit said petals to move from said first position to said second position.

17. The surgical access port of claim 11, further comprising a plastic membrane within said shaft, said plastic membrane being penetrable by a surgical instrument.

18. The surgical access port of claim 17, wherein said plastic membrane seals against a surgical instrument penetrating therethrough.

19. A method of inserting a gel based laparoscopic trocar having a shaft with an upper portion and a lower portion, a rigid gel casing surrounding the upper portion of the shaft, at least two resilient gel filled membranes filling the first portion of the shaft, a plurality of petals surrounding the outside of the lower portion of the shaft, the petals having a first condition in which said petals are elongated along the length of the shaft and a second condition in which portions of the petals are permitted to curl upward and away from the shaft, said method comprising the steps of:
forming a surgical incision in a patient;
spreading the surgical incision;
inserting the lower portion of the gel based laparoscopic trocar into the incision.

20. The method of claim 19, wherein the step of inserting forces the gel based laparoscopic trocar into the incision such that the gel casing abuts an exterior portion of the incision.

21. The method of claim 20, further comprising the step of moving the petals from the first condition to the second condition such that the petals abut an interior portion of the incision.
